(19) United States
(12) Patent Application Publication
(10) Pub. No.: US 2004/0106190 A1
(43) Pub. Date: Jun. 3, 2004

(54) Flow-through assay devices

(75) Inventors: Kaiyuan Yang, Cumming, GA (US); Xuedong Song, Roswell, GA (US); Kevin Peter McGrath, Alpharetta, GA (US); Rameshbabu Boga, Roswell, GA (US); Shawn Ray Feaster, Duluth, GA (US); David Cohen, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/308,926

(22) Filed: Dec. 3, 2002

(51) Int. Cl. C12M 1/34

(52) U.S. Cl. 435/287.2

(57) ABSTRACT

A flow-through assay device capable of detecting the presence or quantity of an analyte of interest is provided. The device is in communication with an electrochemical biosensor that utilizes detection and calibration working electrodes that communicate with affinity reagents, such as redox mediators and capture ligands. For instance, capture ligands that are specific binding members for the analyte of interest may be applied to the detection electrode to serve as the primary location for detection of the analyte. The calibration working electrode may be used to calibrate the detection working electrode for any intrinsic background current not generated by the reagents of the biosensor system. Moreover, capture ligands that are non-specific binding members for the analyte of interest may also be applied to the calibration electrode. In such instances, the calibration electrode may be used to calibrate the detection working electrode for any non-specific binding that may contribute to the current generated on the surface thereof.

### BACKGROUND OF THE INVENTION

Various analytical procedures and devices are commonly employed in assays to determine the presence and/or absence of analytes in a test sample. For instance, immunoassays utilize mechanisms of the immune systems, wherein antibodies are produced in response to the presence of antigens that are pathogenic or foreign to the organisms. These antibodies and antigens, i.e., immunoreactants, are capable of binding with one another, thereby causing a highly specific reaction mechanism that can be used to determine the presence or concentration of that particular antigen in a biological sample. There are several well-known techniques for detecting the presence of an analyte.

One example of such a technique employs the use of an electrochemical affinity biosensor that detects the coupling reaction between a capture ligand immobilized on an electrode surface and an analyte. A number of electrochemical affinity biosensors have been developed. For instance, a biosensor was developed that utilized an alkaline phosphatase antibody conjugate to perform sandwich immunoassays (Xu, and Heineman et al., Clin. Chem, 36, 1941-1944, 1990). In these assays, aminophenyl phosphate was used as a substrate and the aminophenol product was detected anodically with a flow-injection analysis system. Electrochemical affinity biosensors were also developed that utilized enzyme labels. (Bourdillon et al., J. Am Chem. Soc., 115, 1226, 1993).

Unfortunately, one of the problems with such electrochemical affinity biosensors was that they required washing and separation steps. These two steps caused irreproducibility and resulted in extra steps for performing the assay. Thus, a biosensor was proposed in which the substrate was converted into an electroactive product and brought into the cell from behind the electrode. (Duan, et al., Anal. Chem., 66, 1369, 1994). This approach allowed for measurement of the binding reaction without the usual washing steps. However, the cell and electrodes had to be specifically designed for each potential application.

Still another biosensor involved the detection of H₂O₂ by covalently immobilizing horse radish peroxidase (HRP) in a redox hydrogel. (Vreeks, et al., Anal. Chem., 64, 3084, 1992; also Anal Chem., 67, 4247, 1995). The redox hydrogel was formed of HRP and water-soluble poly(vinylpyridine), which was quaternized with 2-bromoethylamine and osmium bipyridine redox centers (PVP-NH2-Os), and cross-linked with poly(ethylene glycol diglycidyl ether) on vitreous carbon. In these electrodes, the catalytic electroreduction of H₂O₂ was observed with as little as 1 μg/cm² HRP incorporated in the hydrogel. Modification of the catalytic behavior of the hydrogel by addition of small amounts of HRP led to the hypothesis that the specific binding of HRP-labeled affinity reagents to an electrode could be selectively detected and that the resulting electrochemical affinity biosensors would not require washing or separation of the affinity reagents.

Despite the improvements provided by current biosensors, problems nevertheless remain. For instance, intrinsic background current from the electrodes often affects the accuracy of the measured current and thus the determined analyte concentration. In addition, other current-generating compounds can also become unintentionally bound to the capture ligand on the electrode surface, thereby further affecting the accuracy of the determined analyte concentration. Further, many of the current electrochemical sensors are impractical and too expensive for many applications, such as point-of-care or over-the-counter applications

As such, a need still exits for sensors that are more reliable, practical, and cost effective.

### SUMMARY OF THE INVENTION

In accordance with one embodiment of the present invention, a flow-through assay device (e.g., membrane-based, fluidics-based, and so forth) is disclosed for detecting the presence or quantity of an analyte residing in a test sample. The flow-through assay device comprises a fluidic medium (e.g., porous membrane, channel, etc.). In some embodiments, a redox label is applied to the fluidic medium for directly or indirectly binding to the analyte. The redox label, for instance, can be an enzyme selected from the group consisting of alkaline phosphatase (AP), horseradish peroxidase (HRP), glucose oxidase, beta-galactosidase, urease, and combinations thereof. In one embodiment, the enzyme is horseradish peroxidase formed, for example, using the periodate method. If desired, the redox label can be bound to a specific binding member for the analyte.

The fluidic medium is in communication with an electrochemical affinity biosensor. The biosensor comprises a detection working electrode capable of generating a measurable detection current. A specific binding capture ligand for the analyte is applied to the detection working electrode. For example, the specific binding capture ligand may be selected from the group consisting of antigens, haptens, aptamers, antibodies, and complexes thereof, and may have a specificity for the analyte at concentrations as low as about 10⁻⁹ moles of the analyte per liter of test sample. In addition, a redox mediator may also be applied to the detection working electrode, before and/or after the test sample is applied to the assay device. For example, the redox mediator may be selected from the group consisting of oxygen, ferrocene derivatives, quinones, ascorbic acids, redox polymers with metal complexes, redox hydrogel polymers, and organic compounds.

Besides a detection working electrode, the biosensor also comprises a calibration working electrode capable of generating a measurable calibration current. The calibration working electrode may be formed from substantially the same material and have approximately the same shape and size as the detection working electrode. Further, in some embodiments, a non-specific binding capture ligand and a redox mediator may be applied to the calibration working electrode. For instance, the non-specific binding capture ligand may be selected from the group consisting of antigens, haptens, aptamers, antibodies, and complexes thereof, and may have no specificity for the analyte of interest at concentrations as high as about 10⁻² moles of the analyte per liter of the test sample.

In accordance with another embodiment of the present invention, a method is disclosed for detecting the presence or quantity of an analyte residing in a test sample. The method comprises:

i) providing a flow-through assay device comprising a fluidic medium in communication with an electrochemical affinity biosensor, the biosensor comprising a detection working electrode to which is applied a specific binding capture ligand for the analyte and a calibration working electrode;

ii) contacting a test sample containing the analyte with the fluidic medium;

iii) allowing the test sample to flow through the fluidic medium to contact the detection working electrode and the calibration working electrode;

iv) applying a potential difference, such as with a multi-channel potentiostat, between the detection working electrode and a counter electrode and between the calibration working electrode and a counter electrode;

v) measuring the current generated at the detection working electrode and the current generated at the calibration working electrode;

vi) determining a calibrated detection current by calibrating the current generated at the detection working electrode by the current generated at the calibration working electrode; and

vii) correlating the calibrated detection current to a concentration for the analyte.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a schematic illustration of one embodiment of a flow-through assay device of the present invention;

FIG. 2 is a schematic illustration of another embodiment of a flow-through assay device of the present invention;

FIG. 3 illustrates the “periodate” method of forming a horseradish peroxidase (HRP) conjugate for use in one embodiment of the present invention;

FIG. 4 is a schematic illustration of still another embodiment of a flow-through assay device of the present invention;

FIG. 5 is a graph of the current detected (milliamps) versus the potential applied to each electrode in Example 2;

FIG. 6 is a graph of the current applied (microamps) versus time (seconds) for Example 4; and

FIG. 7 is a graph of the current applied (milliamps) versus time (seconds) for Example 5.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “analyte” generally refers to a substance to be detected. For instance, analytes can include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MIB (CK-MB); digoxin; phenytoin; phenobarbitol; carbamazepine; vancomycin; gentamycin; theophylline; valproic acid; quinidine; leutinizing hormone (LH); follicle stimulating hormone (FSH); estradiol, progesterone; C-reactive protein; lipocalins; IgE antibodies; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-lgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); thyroid stimulating hormone (TSH); thyroxine (T4); total triiodothyronine (Total T3); free triiodothyronine (Free T3); carcinoembryoic antigen (CEA); and alpha fetal protein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651 to Everhart, et al. and U.S. Pat. No. 4,366,241 to Tom et al.

As used herein, the term “test sample” generally refers to a material suspected of containing the analyte. The test sample can be used directly as obtained from the source or following a pretreatment to modify the character of the sample. The test sample can be derived from any biological source, such as a physiological fluid, including, blood, saliva, mucous, ocular lens fluid, cerebral spinal fluid, sweat, urine, milk, ascites fluid, raucous, synovial fluid, peritoneal fluid, vaginal fluid, seminal fluid, amniotic fluid or the like. The test sample can be pretreated prior to use, such as preparing plasma from blood, diluting viscous fluids, and so forth. Methods of treatment can involve filtration, distillation, concentration, inactivation of interfering components, and the addition of reagents. Besides physiological fluids, other liquid samples can be used such as water, food products for the performance of environmental or food production assays. In addition, a solid material suspected of containing the analyte can be used as the test sample. In some instances it may be beneficial to modify a solid test sample to form a liquid medium or to release the analyte.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents.

In general, the present invention is directed to a flow-through assay device capable of detecting the presence or quantity of an analyte of interest. The device is in communication with an electrochemical biosensor that is accurate, reliable, and easy-to-use. In particular, the biosensor utilizes detection and calibration working electrodes that communicate with affinity reagents, such as redox mediators and capture ligands. For instance, capture ligands that are specific binding members for the analyte of interest are applied to the detection electrode to serve as the primary location for detection of the analyte. The calibration working electrode may be used to calibrate the detection working electrode for any intrinsic background current not generated by the reagents of the biosensor system. Moreover, capture ligands that are non-specific binding members for the analyte of interest may also be applied to the calibration electrode. In such instances, the calibration electrode may be used to calibrate the detection working electrode for any non-specific binding that may contribute to the current generated on the surface thereof.

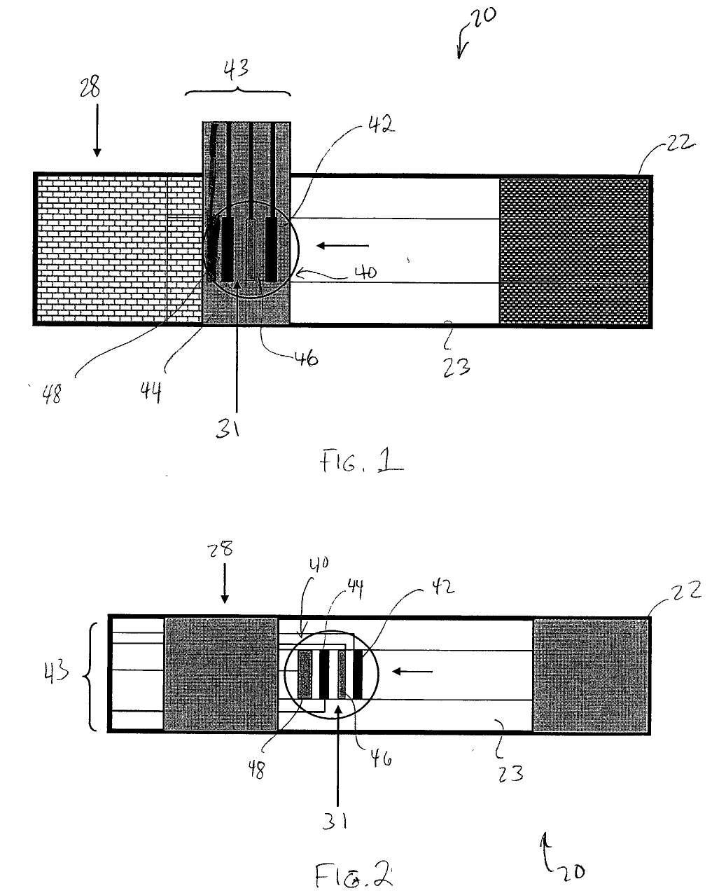

Referring to FIG. 1, for instance, one embodiment of a membrane-based flow-through assay device 20 that can be formed according to the present invention will now be described in more detail. As shown, the device 20 contains a porous membrane or mesh 23 that acts as a fluidic medium and is optionally supported by a rigid material (not shown). In general, the porous membrane 23 can be made from any of a variety of materials through which the test sample is capable of passing. For example, the materials used to form the porous membrane 23 can include, but are not limited to, natural, synthetic, or naturally occurring materials that are synthetically modified, such as polysaccharides (e.g., cellulose materials such as paper and cellulose derivative's, such as cellulose acetate and nitrocellulose); silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the porous membrane 23 is formed from nitrocellulose and/or polyester sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms. In another embodiment, the membrane 23 may be a mesh-type membrane, such as nylon mesh membranes that are commercially available from Millipore Corporation.

The device 20 may also contain a wicking pad 28. The wicking pad 28 generally receives fluid that has migrated through the entire porous membrane 23. As is well known in the art, the wicking pad 28 can assist in promoting capillary action and fluid flow through the membrane 23.

To initiate the detection of an analyte within the test sample, a user may directly apply the test sample to a portion of the porous membrane 23 through which it can then travel. Alternatively, the test sample may first be applied to a sampling pad (not shown) that is in fluid communication with the porous membrane 23. Some suitable materials that can be used to form the sampling pad include, but are not limited to, nitrocellulose, cellulose, porous polyethylene pads, and glass fiber filter paper. If desired, the sampling pad may also contain one or more assay pretreatment reagents, either diffusively or non-diffusively attached thereto. In the illustrated embodiment, the test sample travels from the sampling pad (not shown) to a conjugate pad 22 that is placed in communication with one end of the sampling pad. The conjugate pad 22 is formed from a material through which the test sample is capable of passing. For example, in one embodiment, the conjugate pad 22 is formed from glass fibers. Although only one conjugate pad 22 is shown, it should be understood that other conjugate pads may also be used in the present invention.

Although the analyte of interest may be inherently capable of undergoing the desired oxidation/reduction reactions because it contains a redox center, it may be desired, in other embodiments, to attach a redox label to the analyte. The redox label may be applied at various locations of the device 20, such as to the conjugate pad 22, where it may bind to the analyte of interest. Alternatively, the analyte may be bound to a redox label prior to being applied to the device 20. The term “redox label” refers to a compound that has one or more chemical functionalities (i.e., redox centers) that can be oxidized and reduced. Such redox labels are well known in the art and may include, for instance, an enzyme such as alkaline phosphatase (AP), horseradish peroxidase (HRP), glucose oxidase, beta-galactosidase, urease, and so forth. Other organic and inorganic redox compounds are described in U.S. Pat. No. 5,508,171 to Walling, et al.; U.S. Pat. No. 5,534,132 to Vreeke, et al.; U.S. Pat. No. 6,241,863 to Monbouguefte; and U.S. Pat. No. 6,281,006 to Heller, et al., which are incorporated herein in their entirety by reference thereto for all purposes.

Horseradish peroxidase (HRP), for instance, is an enzyme that is commonly employed in electrochemical affinity biosensors. Two methods are commonly used for the preparation of antibody-coupled horseradish peroxidase (HRP) conjugates, i.e., “glutaraldehyde” and “periodate” oxidation. As is known in the art, the “glutaraladehyde” method involves two steps and results in high molecular weight aggregates. Further, the “periodate” method involves three steps. For instance, as shown in FIG. 3, the “periodate” method may reduce interference of HRP active-site amino groups because it is only conjugated through carbohydrate moieties. Specifically, the “periodate” method opens up the carbohydrate moiety of the HRP glycoprotein molecule to form aldehyde groups that will form Schiff bases with antibody amino groups. Thus, although not required, it may be desired to use HRP formed by the “periodate” method to minimize background current.

Besides being directly attached to the analyte, the redox label may also be indirectly attached to the analyte through a specific binding member for the analyte. Specific binding members generally refer to a member of a specific binding pair, i.e., two different molecules where one of the molecules chemically and/or physically binds to the second molecule. For instance, immunoreactive specific binding members can include antigens, haptens, aptamers, antibodies, and complexes thereof, including those formed by recombinant DNA methods or peptide synthesis. An antibody can be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin, carbohydrates and lectins, complementary nucleotide sequences (including label and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs can include members that are analogs of the original specific binding member. For example, a derivative or fragment of the analyte, i.e., an analyte-analog, can be used so long as it has at least one epitope in common with the analyte.

The redox labels may be used in a variety of ways to form a probe. For example, the labels may be used alone to form probes. Alternatively, the labels may be used in conjunction with polymers, liposomes, dendrimers, and other micro- or nano-scale structures to form probes. In addition, the labels may be used in conjunction with microparticles (sometimes referred to as “beads” or “microbeads”) to form probes. For instance, naturally occurring microparticles, such as nuclei, mycoplasma, plasmids, plastids, mammalian cells (e.g., erythrocyte ghosts), unicellular microorganisms (e.g., bacteria), polysaccharides (e.g., agarose), and so forth, can be used. Further, synthetic microparticles may also be utilized. For example, in one embodiment, latex microparticles are utilized. Although any latex microparticle may be used in the present invention, the latex microparticles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchlorideacrylates, and so forth, or an aldehyde, carboxyl, amino, hydroxyl, or hydrazide derivative thereof. Other suitable microparticles may be described in U.S. Pat. No. 5,670,381 to Jou, et al. and U.S. Pat. No. 5,252,459 to Tarcha, et al., which are incorporated herein in their entirety by reference thereto for all purposes. In addition, inorganic particles, such as colloidal metallic and non-metallic particles (e.g., gold), carbon particles, and the like, may also be utilized.

When particles are utilized, such as described above, the mean diameter of the particles may generally vary as desired depending on factors such as the type of particle chosen, the pore size of the membrane, and the membrane composition. For example, in some embodiments, the mean diameter of the particulate labels can range from about 0.01 microns to about 1,000 microns, in some embodiments from about 0.01 microns to about 100 microns, and in some embodiments, from about 0.01 microns to about 10 microns. In one particular embodiment, the particles have a mean diameter of from about 0.01 to about 2 microns. Generally, the particles are substantially spherical in shape, although other shapes including, but not limited to, plates, rods, bars, irregular shapes, etc., are suitable for use in the present invention. As will be appreciated by those skilled in the art, the composition, shape, size, and/or density of the particles may widely vary.

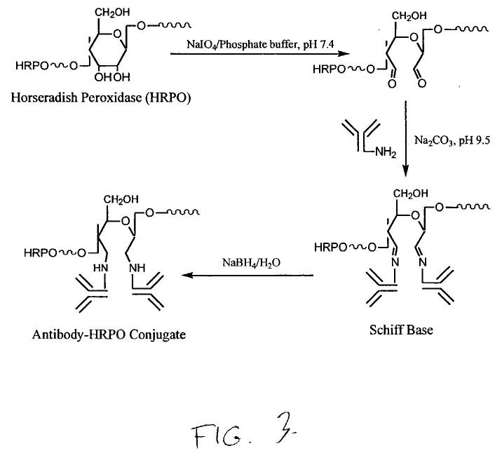

Once labeled, if desired, the analyte of interest may then travel through the porous membrane 23 until it reaches a detection zone 31. At the detection zone 31, the analyte contacts an electrochemical biosensor strip 40. As shown in FIG. 1, the strip 40 may be laminated to the porous membrane 23 adjacent to the wicking pad 28. In this embodiment, the leads 43 for the strip 40 are disposed perpendicular to the flow of the test sample. Alternatively, as shown in FIG. 2, the strip 40 may be positioned so that the leads 43 are parallel to the flow of the test sample.

Typically, the strip 40 is formed from an insulative substrate, such as silicon, fused silicon dioxide, silicate glass, alumina, aluminosilicate ceramic, an epoxy, an epoxy composite such as glass fiber reinforced epoxy, polyester, polyimide, polyamide, polycarbonate, etc. Various electrodes are formed on the substrate of the strip 40. Specifically, as shown, a detection working electrode 42, a calibration working electrode 44, a counter electrode 46, and a reference electrode 48, are formed on the substrate of the strip 40. These electrodes may be positioned at any angle to the flow of the test sample through the porous membrane 23. The reference and counter electrodes 46 and 48 may be combined into a single “pseudo” electrode. This may be particularly beneficial when the solution resistance is negligible or the generated current is relatively small. Moreover, it should be understood that each working electrode 42 and 44 may be paired with a separate counter and reference electrode. Further, multiple detection and calibration working electrodes 42 and 44 may be utilized.

The detection working electrode 42 is typically formed from a thin film of conductive material disposed on the insulating substrate of the strip 40. Generally speaking, a variety of conductive materials can be used to form the detection working electrode 42. Suitable materials include, for example, carbon, metals, metal-based compounds (e.g., oxides, chlorides, etc.), metal alloys, conductive polymers, combinations thereof, and so forth. Examples of carbon electrodes include glassy carbon, graphite, mesoporous carbon, nanocarbon tubes, fullerenes, etc. Commercially available carbon paste from DuPont or other vendors are also suitable for the current invention. Examples of metals suitable for the current invention include platinum, palladium, gold, tungsten, titanium, etc, and their alloys. Certain metal paste compositions may also be used for the construction of the working electrodes. Thin films of these materials can be formed by a variety of methods including, for example, sputtering, reactive sputtering, physical vapor deposition, plasma deposition, chemical vapor deposition, printing, and other coating methods. For instance, carbon or metal paste based conductive materials are typically formed using screen printing, which either can be done manually or automatically. Likewise, metal-based electrodes are typically formed using standard sputtering or CVD techniques, or by electrochemical plating.

Discrete conductive elements may be deposited to form each of the detection working electrode 42, for example, using a patterned mask. Alternatively, a continuous conductive film may be applied to the substrate and then the detection working electrode 42 can be patterned from the film. Patterning techniques for thin films of metal and other materials are well known in the semiconductor art and include photolithographic techniques. An exemplary technique includes depositing the thin film of conductive material and then depositing a layer of a photoresist over the thin film. Typical photoresists are chemicals, such as organic compounds, that are altered by exposure to light of a particular wavelength or range of wavelengths. Exposure to light makes the photoresist either more or less susceptible to removal by chemical agents. After the layer of photoresist is applied, it is exposed to light, or other electromagnetic radiation, through a mask. Alternatively, the photoresist is patterned under a beam of charged particles, such as electrons. The mask may be a positive or negative mask depending on the nature of the photoresist. The mask includes the desired pattern of working electrodes, which are the electrodes on which the electrocatalytic reactions take place when the detection marker and the redox label are both present and immobilized on the electrode. Once exposed, the portions of the photoresist and the thin film between the working electrodes is selectively removed using, for example, standard etching techniques (dry or wet), to leave the isolated working electrodes of the array.

The detection working electrode 42 can have a variety of shapes, including, for example, square, rectangular, circular, ovoid, and so forth. The detection working electrode 42 may have varying dimensions (e.g., length, width, or diameter), such as from about 50 micrometers to about 5 millimeters. In some embodiments, the detection working electrode 42 is a three-dimensional structure, and can have a surface area of from about 1×10⁻⁴ square centimeters to about 0.25 square centimeters. The surface smoothness and layer thickness of the electrode 42 may be controlled through a combination of a variety of parameters, such as mesh size, mesh angle, and emulsion thickness when using a printing screen. Emulsion thickness can be varied to adjust wet print thickness. The dried thickness may be slightly less than the wet thickness because of the vaporization of solvents. In some embodiments, for instance, the dried thickness of the printed electrode 42 is less than about 100 microns, in some embodiments less than about 50 microns, in some embodiments less than about 20 microns, in some embodiments less than about 10 microns, and in some embodiments, less than about 1 micron.

In addition, one or more surfaces of the detection working electrode 42 are generally treated with various affinity reagents. For example, in one embodiment, the surface of the detection working electrode 42 is treated with a specific binding capture ligand. The specific binding capture binding ligand is capable of directly or indirectly binding to the analyte of interest. The specific binding capture ligand typically has a specificity for the analyte of interest at concentrations as low as about 10⁻⁷ moles of the analyte per liter of test sample (moles/liter), in some embodiments as low as about 10⁻⁸ moles/liter, and in some embodiments, as low as about 10⁻⁹ moles/liter. For instance, some suitable immunoreactive specific binding capture ligands can include antigens, haptens, aptamers, antibodies, and complexes thereof, including those formed by recombinant DNA methods or peptide synthesis. Generally speaking, electrochemical stability is desired for accurate analyte detection because any redox response from the specific binding capture ligand may complicate the true current responses from the analyte. Thus, in most embodiments, the specific binding capture ligand is stable at the potential range of from −0.75 to +0.75 Volts, in some embodiments from −0.50 to +0.50 Volts, and in some embodiments, from −0.35 to +0.35 Volts, in comparison with the reference electrode.

Besides specific binding capture ligands, redox mediators may also be applied to the surface of the detection working electrode 42. The redox mediators may be applied to the working electrode 42 at any time, such as during formation of the assay device or during testing. In one embodiment, for instance, the redox mediator is immobilized on the surface of the electrode 42. Alternatively, in another embodiment, the redox mediator is applied to the surface only after the test sample reaches the detection zone 31. Some examples of suitable redox mediators that can be used in the present invention include, but are not limited to, oxygen, ferrocene derivatives, quinones, ascorbic acids, redox polymers with metal complexes, glucose, redox hydrogel polymers, and organic compounds. Particular examples of suitable redox mediators include ferricyanide, 2,5-dichloro-1,4-benzoquinone, 2,6-dichloro-1,4-benzoquinone, 2,6-dimethyl-1,4-benzoquinone, phenazine ethosulfate, phenazine methosulfate, phenylenediamine, 1-methoxy-phenazine methosulfate, and 3,3′5,5′ tetramethyl benzidine (TMB). Substrates may also be used in conjunction with a soluble mediator present in solution. In such instances, the solution-containing substrate may be simply placed on the surface of the applicable electrode. Some commercially available examples of such solution-containing substrates include 1-Step turbo TMB (Pierce Chemical Co., Rockford, Ill.) and K-Blue Substrate Ready-to-Use (Neogen Corp., Lexington, Ky.). For instance, “K-Blue Substrate” is a chromogenic substrate for horseradish peroxidase that contains 3,3′,5,5′ tetramethylbenzidine (TMB) and hydrogen peroxide (H₂O₂). Other suitable redox mediators are described in U.S. Pat. No. 6,281,006 to Heller, et al.; U.S. Pat. No. 5,508,171 to Walling, et al.; U.S. Pat. No. 6,080,391 to Tsuchiya, et al.; and U.S. Pat. No. 6,461,496 to Feldman, et al., which are incorporated herein in their entirety by reference thereto for all purposes.

The affinity reagents may be applied to the surface of the detection working electrode 42 using a variety of well-known techniques. For example, the reagents may be directly immobilized on the surface of the electrode 42, may be contained within a substrate that is disposed on the surface of the electrode 42, may be mixed into the materials used to form the electrode 42, and so forth. In one embodiment, the affinity reagents are formulated into a solution and screen-printed, ink-jet printed, drop coated, or sprayed onto the working electrode surface. Screen printing inks, for instance, are typically formulated in a buffer solution (e.g., phosphate buffer) containing the specific or non-specific binding members. Although not required, an organic immobilizing solvent can be added to the buffer solution to help wet the hydrophobic or non-hydrophilic surfaces. In some embodiments, for example, the solvent can be an alcohol, ether, ester, ketone, or combinations thereof. When coated, the electrode 42 is desirably applied with a uniform coating across its entire surface. The coating is typically a single layer, but multiple layers are also contemplated by the present invention. The coating, regardless of monolayer or multiple layers, is typically optimized to give the largest current and signal/noise ratio.

Upon application to the electrode surface, the reagents can optionally be stabilized. Stabilization facilitates long-term stability, particularly for ensuring required shelf-life during incurred during shipping and commercial selling. For instance, in one embodiment, stabilization can be accomplished by coating a layer, such as a polymer, gel, carbohydrate, etc., onto the electrode surface before and/or after application of the affinity reagent(s). Some commercially available examples of such a stabilization coating are Stabilcoat®, Stabilguard®, and Stabilzyme® from Surmodics, Inc. of Eden Prairie, Minn.

Besides a detection working electrode 42, the biosensor strip 40 also includes a calibration working electrode 44. The calibration working electrode 44 can enhance the accuracy of the analyte concentration determination in a variety of ways. For instance, a current will generally be generated at the calibration working electrode 44 that corresponds to intrinsic background interference stemming from the counter and reference electrodes, as well as the working electrodes themselves. Once determined, the value of this intrinsic background current can be used to calibrate the measured current value at the detection working electrode 42 to obtain a more accurate reading. The calibration working electrode 44 may generally be formed as described above with respect to the detection working electrode 42. In fact, because the calibration working electrode 44 is configured to calibrate the detection working electrode 42, it is generally desired that such electrodes are formed in approximately the same manner, from the same materials, and to have the same shape and/or size.

The detection and calibration working electrodes 42 and 44 are also generally applied with the same surface treatments to improve the calibration accuracy. However, one primary difference between the detection working electrode 42 and the calibration working electrode 44 is that the electrode 44 does not typically contain a specific binding capture ligand for the analyte of interest. This allows most if not all of the analyte to bind to the electrode 42, thereby enabling the electrode 42 to be used primarily for detection and the electrode 44 to be used primarily for calibration.

In some instances, non-specific binding of the redox label or other current-generating compounds to the capture ligand present on the detection working electrode 42 may create inaccuracies in the measured current. Thus, if desired, non-specific binding capture ligands may be applied to one or more surfaces of the calibration working electrode 44. Similar to the specific binding capture ligands described above, the non-specific binding capture ligands may also include, for instance, antigens, haptens, aptamers, antibodies, and complexes thereof. However, contrary to the specific binding ligands, the non-specific binding ligands do not have a high specificity for the analyte of interest. In fact, the non-specific binding capture ligand typically has no specificity for the analyte of interest at concentrations as high as about 10⁻² moles of the analyte per liter of test sample (moles/liter), and in some embodiments, as high as about 10⁻³ moles/liter. The non-specific binding ligands can form bonds with various immunoreactive compounds. These immunoreactive compounds may have a redox center or may have inadvertently been provided with a redox center through attachment of a redox compound (e.g., enzyme). Without the calibration working electrode 44, these immunoreactive compounds would thus generate a low level of current, which causes error in the resulting analyte concentration calculated from the generated current. This error may be substantial, particularly when the test sample contains a low analyte concentration.

To minimize any undesired binding on the surfaces of the working electrodes 42 and 44, the counter electrode 46, or the reference electrode 48, a blocking agent may be applied thereto. The term “blocking agent” means a reagent that adheres to the electrode surface so that it “blocks” or prevents certain materials from binding to the surface. Blocking agents can include, but are not limited to, β-casein, albumins such as bovine serum albumin, gelatin, pluronic or other surfactants, polyethylene glycol, polyvinyl alcohol, polyvinyl pyrrolidinone or sulfur derivatives of the above compounds, a surfactant such as Tween 20, 30, 40 or Triton X-100, a polymer such as polyvinyl alcohols, and any other blocking material known to those of ordinary skill in the art. Depending on the conductive materials used for preparing the working electrodes, the blocking agents may be formulated to adapt to the electrode surface properties. In some embodiments, a cocktail containing multiple blocking agents can be applied onto an electrode and incubated for 5 to 30 minutes, then any excess solution can be removed and the resulting electrode thoroughly dried.

In general, a variety of assay formats may be used in the present invention. In this regard, various embodiments of the present invention will now be described in more detail. It should be understood, however, that the embodiments discussed below are only exemplary, and that other embodiments are also contemplated by the present invention. For instance, referring again to FIG. 1, a test sample containing an analyte can initially be applied to the sampling pad. From the sampling pad, the test sample can then travel to the conjugate pad 22, where the analyte mixes and attaches to a redox label. In one embodiment, for instance, the label is horseradish peroxidase (HRP) and the analyte of interest is glucose. Because the conjugate pad 22 is in fluid communication with the porous membrane 23, the labeled analyte can migrate from the conjugate pad 22 to a detection zone 31 present on the porous membrane 23, where it contacts the biosensor strip 40. The labeled analyte binds to the specific binding capture ligand on the detection working electrode 42 where it reacts with a redox mediator. In one embodiment, for example, the analyte is reacted as follows:

Analyte(reduced form)+Redox Mediator(oxidized form)→Analyte(oxidized form)+Redox Mediator(reduced form)

In addition, non-analyte biological materials may also bind to the non-specific binding capture ligand on the calibration working electrode 44 where it reacts with a redox mediator. It is intended that the amount of non-analyte materials that bind to the calibration working electrode 44 will be similar to the amount of non-analyte material that non-specifically binds to the detection working electrode 42. Thus, in this manner, the background signal due to non-specific binding can be compensated. In one embodiment, for example, the non-analyte biological materials (abbreviated “NAB”) are reacted as follows:

NAB(reduced form)+Redox Mediator(oxidized form)→NAB(oxidized form)+Redox Mediator(reduced form)

After the reactions are complete, a potentiostat applies a potential difference between the detection working electrode 42 and counter electrode 46. When the potential difference is applied, the amount of the oxidized form of the redox mediator at the counter electrode 46 and the potential difference is sufficient to cause diffusion limited electrooxidation of the reduced form of the redox mediator at the surface of the detection working electrode 42. The diffusion limited current generated by the oxidation of the reduced form of the redox mediator at the surface of the calibration detection working electrode 42. Similarly, the potential difference is also supplied between the calibration working electrode 44 and counter electrode 46. When the potential difference is applied, diffusion limited electrooxidation of the reduced form of the redox mediator at the surface of the calibration working electrode 44. Again, the diffusion limited current generated by the oxidation of the reduced form of the redox mediator at the surface of the calibration working electrode 44.

Generally, the detection and calibration working electrodes 42 and 44 simultaneously generate a respective signal from a single measurement of a sample. The simultaneously generated signals are averaged by a processing circuit, such as a multi-channel potentiostat. Multi-channel potentiostats are well known in the art, and are described, for instance, in U.S. Pat. No. 5,672,256 to Yee, which is incorporated herein in its entirety by reference thereto for all purposes. Each channel of a multi-channel potentiostat can function as a potentiostat, and thus may be associated with its own reference and/or counter electrode, or may share reference and/or counter electrodes. One suitable example of a multi-channel potentiostat that may be used in the present invention is commercially available under the name “MSTAT” from Arbin Instruments, Inc. of College Station, Tex. Once detected, the current measured at the detection working electrode 42 is calibrated by the current measured at the electrode 44 to obtain a calibrated current reading that may be correlated to the concentration of analyte in the sample. The correlation may result from predetermined empirical data or an algorithm, as is well known in the art. If desired, the generated current and analyte concentration may be plotted as a curve to aid in the correlation therebetween. As a result, calibration and sample testing may be conducted under approximately the same conditions at the same time, thus providing reliable quantitative or semi-quantitative results, with increased sensitivity. In the case of a sandwich assay format, the signal provided by the detection working electrode 42 is directly proportional to the analyte concentration in the test sample. In the case of a competitive assay format, which may, for instance, be constructed by applying a labeled analyte on the surface of the detection working electrode 42, the signal provided by the detection working electrode 42 is inversely proportional to the analyte concentration in the test sample.

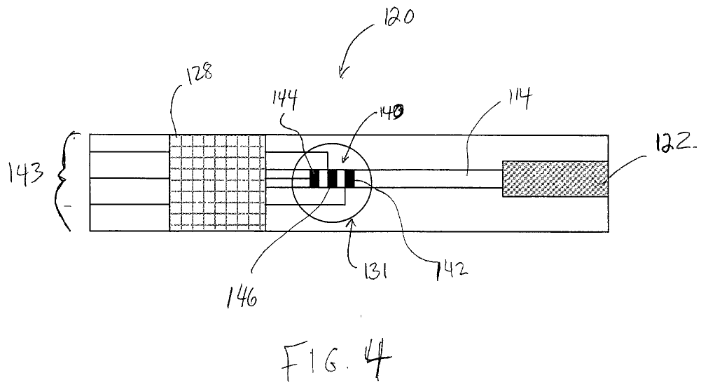

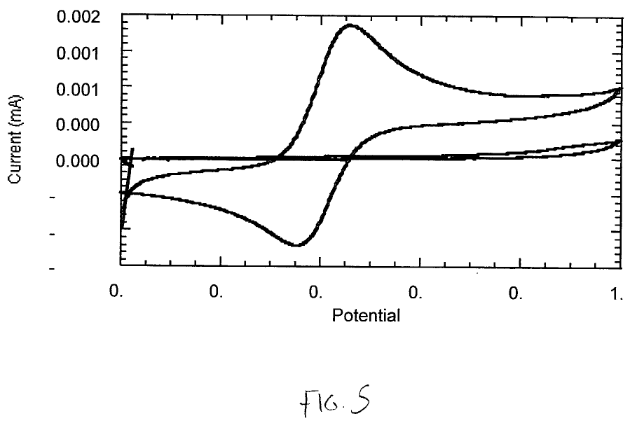

Besides flow-through devices that utilize a porous membrane as a fluidic medium, such as described above, other flow-through devices may also be formed according to the present invention. For example, referring to FIG. 4, one embodiment of a fluidics-based device 120 that may be formed according to the present invention is illustrated. As shown, the device 120 has a chamber 122 in fluid communication with a fluidic channel 114, that together function as a fluidic medium for the test sample. Although the chamber 122 and the fluidic channel 114 are shown in a substantial linear relationship, it should be understood that the chamber 122 and channel 114 may also be disposed in other relationships as well. Further, it should also be understood that the chamber 122 and the fluidic channel 114 may be the same or different. For instance, in one embodiment, the fluidic channel 114 and reaction chamber 122 may both be defined by one fluidic cavity.

As described above, a redox label may be applied at various locations of the device 120, such as to the chamber 122 or the channel 114, where it may bind to the analyte of interest. If desired, the geometry of the fluidic channel 114 may be selected so that capillary forces assist the flow of the test sample from the chamber 122 to the fluidic channel 114. For example, in some embodiments, the fluidic channel 114 may have a width that is from about 1 micrometer to about 5 centimeters, and in some embodiments, from about 50 micrometers to about 500 micrometers. Further, the fluidic channel 114 may have a length direction that is from about 1 millimeter to about 50 centimeters, and in some embodiments, from about 10 millimeters to about 50 millimeters. The fluidic channel 114 may also have a height that is from about 0.025 micrometers to about 50 millimeters, and in some embodiments, from about 5 micrometers to about 500 micrometers.

Once labeled, if desired, the analyte of interest may then travel through the fluidic channel 114 until it reaches a detection zone 131. At the detection zone 131, the analyte contacts an electrochemical biosensor strip 140. As shown in FIG. 3, the strip 140 may be disposed within the fluidic channel 114 adjacent to a wicking pad 128. In this embodiment, the leads 143 for the strip 140 are disposed parallel to the flow of the test sample, and a detection working electrode 142, a calibration working electrode 144, and a counter/reference electrode 146 are formed on the substrate of the strip 140. The presence of the analyte may then be determined as set forth above.

Although various embodiments of assay formats and devices have been described above, it should be understood, that the present invention may utilize any assay format or device desired, and need not contain all of the components described above. Further, other well-known components of assay formats or devices not specifically referred to herein may also be utilized in the present invention. For example, various assay formats and/or devices are described in U.S. Pat. No. 5,508,171 to Walling, et al.; U.S. Pat. No. 5,534,132 to Vreeke, et al.; U.S. Pat. No. 6,241,863 to Monbouguette; U.S. Pat. No. 6,270,637 to Crismore, et al.; U.S. Pat. No. 6,281,006 to Heller, et al.; and U.S. Pat. No. 6,461,496 to Feldman, et al., which are incorporated herein in their entirety by reference thereto for all purposes. In addition, it should be understood that both sandwich and competitive assay formats may be formed according to the present invention. Techniques and configurations of sandwich and competitive assay formats are well known to those skilled in the art.

The present invention provides a low-cost, flow-through assay device that can provide accurate analyte detection. The biosensors of the present invention can be produced as a single test for detecting an analyte or it can be formatted as a multiple test device. The uses for the biosensors of the present invention include, but are not limited to, detection of chemical or biological contamination in garments, such as diapers, the detection of contamination by microorganisms in prepacked foods such as fruit juices or other beverages, and the use of the biosensors of the present invention in health diagnostic applications such as diagnostic kits for the detection of antigens, microorganisms, and blood constituents. It should be appreciated that the present invention is not limited to any particular use or application.

The present invention may be better understood with reference to the following examples.

### EXAMPLE 1

The ability to form various detection and calibration working electrodes was demonstrated. Carbon (7101 or 7102), silver (5000), and silver/silver chloride (5847) inks were obtained from DuPont Biosensor group (Research Triangle Park, North Carolina). A screen frame was first fixed onto a screen frame holder and adjusted according to the printing substrate (Mylar® plastics from DuPont). The working and counter electrodes were printed from carbon inks and reference electrode was printed from silver/silver chloride ink. In order to enhance the conductivity between the printed leads and electrodes, a silver ink liner was first printed underneath of the carbon ink. The insulation of conductive leads from lateral flow membrane was achieved by printing a layer of dielectric ink such as UV curable dielectric ink (5018G, DuPont).

Sheets of the printed substrates were placed at room temperature for 2 hours and then heated at 37° C. for 2 hours. The temperature was then raised to 60° C. and dried an additional 2 hours before again raising the temperature to 120-140° C. for 20 minutes. Such stepwise drying helped achieve high uniformity of the electrode surface, while also removing residue solvents of the original ink formulations. The dried electrodes were then kept either in a plastic bag or in a desiccator.

### EXAMPLE 2

The ability to detect current with detection and calibration working electrodes was demonstrated. Initially, carbon electrodes (Mylar® plastic as substrate) were prepared as set forth in Example 1. A 2-microliter suspension of ferrocene dicarboxylic acid (0.2 milligrams per milliliter of PBS buffer), obtained from Aldrich, was then coated on the detection working electrode while the calibration working electrode remained untreated. The electrodes were then dried and their current responses were recorded simultaneously in a PBS buffer or 0.1 molar potassium chloride solution via cyclic voltammetry.

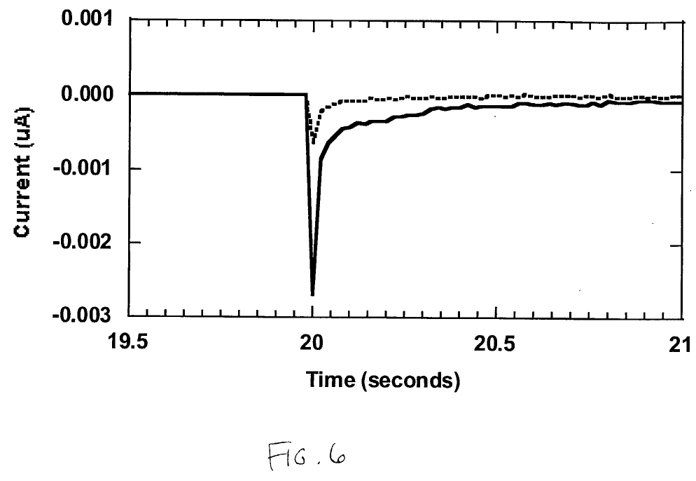

The results are shown in FIG. 5. The upper curve generated by the detection working electrode had a higher signal than the lower curve generated by the calibration working electrode.

### EXAMPLE 3

The ability to form an HRP-conjugated antibody was demonstrated. 2.5 milligrams of HRP were suspended in 0.6 milliliters of water. A solution of 0.15 milliliters of freshly prepared 0.1 molar sodium periodate in 10 millimolar sodium phosphate (pH 7.0) was added to the HRP. The mixture was incubated at room temperature for 20 minutes and dialyzed in 1 millimolar sodium acetate (pH 4.0) at 4° C. using a Pierce cassette dialyzer for several changes. Then, the dialyzed HRP solution (300 microliters) was mixed with 1 milligram of antibody (CRP Mab1 or CRP Mab2) in 100 microliters of a 20 millimolar sodium carbonate (pH 9.5) solution, and incubated at room temperature for 2 hours to form a Schiffs base. The Schiffs base was reduced with 65 microliters of sodium borohydride (2 milligrams per milliliter in water) solution, and incubated at 4° C. for 2 hours. The resulting solution was dialyzed in 10 millimolar PBS buffer for several changes. The antibody-HRP conjugates were stored at 4° C. The above steps involved in HRP conjugation are shown generally in FIG. 3.

### EXAMPLE 4

The ability to detect current with detection and calibration working electrodes was demonstrated. Initially, carbon electrodes were prepared as set forth in Example 2 with Mylar® plastic as the substrate. The detection working electrode was coated with 500 picograms of HRP conjugated LH-β-monoclonal antibody (formed as set forth above in Example 3) and the calibration working electrode was coated with 200 picograms of LH-β-monoclonal antibody. The dried electrodes were treated with excess “1-step Turbo” TMB solution and their current was simultaneously recorded by amperometric measurement at approximately 300 millivolts and 20 seconds after the addition of TMB.

The results are shown in FIG. 6. The upper curve generated by the detection working electrode had a higher signal than the lower curve generated by the calibration working electrode.

### EXAMPLE 5

The ability to form a flow-through assay device in accordance with the present invention was demonstrated. Initially, an electrode strip (carbon working electrode, silver/silver chloride counter/reference electrode, carbon calibration electrode, and Mylar® plastic as backing) was provided. 2 microliters of a capture LH-β-monoclonal antibody solution (about 20 nanograms per milliliter in pH 7.4 PBS buffer) was drop coated onto the detection working electrode surface with an Eppendorf microliter pipette. The resulting electrode strip was then placed at room temperature and air dried. The coated working electrode was then treated with 2 microliters of a protein stabilizing formulation (20 wt. % Stabilcoat™ from Surmodics and 0.05 wt. % Tween 20 in pH 7.4 PBS buffer). The incubation time was 15 minutes. After incubation, the solution was removed by a wicking material and the electrode strip was dried by an air stream. In a similar fashion, the calibration working electrode was coated with a cocktail of blocking agents in pH 7.4 PBS buffer containing casein (1 wt. %) and Tween 20 (0.05 wt. %) and dried.

After treating the electrode strip, a 4.5-centimeter long membrane made of nitrocellulose (Millipore Co.) was laminated thereon. A cellulosic fiber wicking pad (Millipore Co.) was attached to one end of the membrane. The other end of the membrane was laminated with two glass fiber pads (sample and conjugate pads). The conjugate pad and wicking pad were in direct contact with the membrane, and the sample pad was in direct contact with the conjugate pad. The conjugate pad was treated with 3 microliters of LH-a-HRP monoclonal antibody conjugate (20 micrograms per milliliter) and dried for 30 minutes.

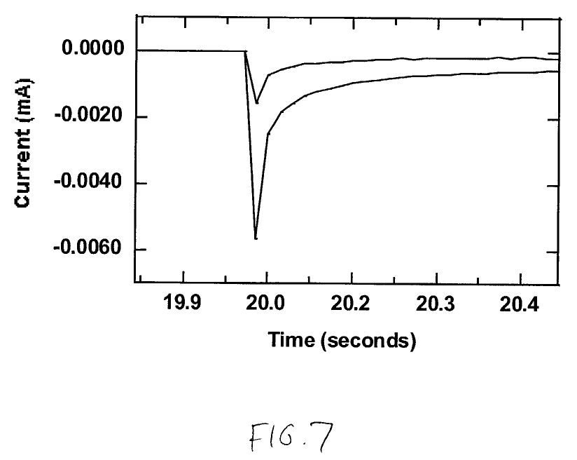

To test the ability of the resulting assay device to detect an analyte, 100 microliters of a sample solution containing LH-antigen (100 nanograms per milliliter) in 10 millimolar PBS buffer (pH of 7.42) was applied to the sample pad. The assay device was allowed to develop until the wicking pad had absorbed almost all of the fluid from the test sample, which occurred in about 5 to 8 minutes. Thereafter, detection was accomplished by directly applying 30 microliters of a TMB substrate solution to the detection zone. The current was recorded after 20 seconds using a multi-channel VMP potentiostat from Perkin Elmer Instruments. The results are shown in FIG. 7. The upper curve generated by the detection working electrode had a higher signal than the lower curve generated by the calibration working electrode.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A flow-through assay device for detecting the presence or quantity of an analyte residing in a test sample, said flow-through assay device comprising a fluidic medium in communication with an electrochemical affinity biosensor, said biosensor comprising:
a detection working electrode capable of generating a measurable detection current, wherein a specific binding capture ligand for the analyte is applied to said detection working electrode; and
a calibration working electrode capable of generating a measurable calibration current, wherein the amount of the analyte within the test sample is determined by calibration of the detection current with the calibration current.

2. A flow-through assay device as defined in claim 1, wherein a redox label is applied to said fluidic medium for directly or indirectly binding to the analyte.

3. A flow-through assay device as defined in claim 2, wherein said redox label is an enzyme.

4. A flow-through assay device as defined in claim 3, wherein said enzyme is selected from the group consisting of alkaline phosphatase, horseradish peroxidase, glucose oxidase, beta-galactosidase, urease, and combinations thereof.

5. A flow-through assay device as defined in claim 4, wherein said enzyme is horseradish peroxidase.

6. A flow-through assay device as defined in claim 2, wherein said redox label is used in conjunction with a microparticle, a nanoparticle, a liposome, a dendrimer, a polymer, or combinations thereof.

7. A flow-through assay device as defined in claim 2, wherein said redox label is used in conjunction with a microparticle modified with a specific binding member for the analyte.

8. A flow-through assay device as defined in claim 1, wherein said biosensor contains an insulative substrate on which said detection and calibration working electrodes are formed.

9. A flow-through assay device as defined in claim 1, wherein said detection and calibration working electrodes are formed from a material selected from the group consisting of carbon, metals, metal oxides, alloys of metals or metal oxides, conductive polymers, and combinations thereof.

10. A flow-through assay device as defined in claim 1, wherein said biosensor further comprises a counter electrode, a reference electrode, or combinations thereof.

11. A flow-through assay device as defined in claim 10, wherein a blocking agent is applied to said detection working electrode, said calibration working electrode, said counter electrode, said reference electrode, or combinations thereof.

12. A flow-through assay device as defined in claim 1, wherein said specific binding capture ligand is selected from the group consisting of antigens, haptens, aptamers, antibodies, and complexes thereof.

13. A flow-through assay device as defined in claim 12, wherein said specific binding capture ligand has a specificity for the analyte at concentrations as low as about 10⁻⁹ moles of the analyte per liter of the test sample.

14. A flow-through assay device as defined in claim 1, wherein a redox mediator is applied to said detection working electrode and said calibration working electrode.

15. A flow-through assay device as defined in claim 14, wherein said redox mediator is selected from the group consisting of oxygen, ferrocene derivatives, quinones, ascorbic acids, redox polymers with metal complexes, glucose, redox hydrogel polymers, and organic compounds.

16. A flow-through assay device as defined in claim 1, wherein said calibration working electrode is formed from substantially the same material and has approximately the same shape and size as said detection working electrode.

17. A flow-through assay device as defined in claim 1, wherein a non-specific binding capture ligand is applied to said calibration working electrode.

18. A flow-through assay device as defined in claim 17, wherein said non-specific binding capture ligand is selected from the group consisting of antigens, haptens, aptamers, antibodies, and complexes thereof.

19. A flow-through assay device as defined in claim 18, wherein said non-specific binding capture ligand has no specificity for the analyte at concentrations as high as about 10⁻² moles of the analyte per liter of the test sample.

20. A flow-through assay device as defined in claim 1, wherein the fluidic medium comprises a porous membrane or mesh.

21. A flow-through assay device as defined in claim 1, wherein the fluidic medium comprises a channel.

22. A flow-through assay device for detecting the presence or quantity of an analyte residing in a test sample, said flow-through assay device comprising a fluidic medium, wherein a redox label is applied to said fluidic medium for directly or indirectly binding to the analyte, said fluidic medium being in communication with an electrochemical affinity biosensor strip, said biosensor strip comprising an insulative substrate on which a detection working electrode capable of generating a measurable detection current and a calibration working electrode capable of generating a measurable calibration current are formed, wherein a specific binding capture ligand for the analyte is immobilized on said detection working electrode, the amount of the analyte within the test sample being determined by calibration of the detection current with the calibration current.

23. A flow-through assay device as defined in claim 22, wherein said redox label is an enzyme selected from the group consisting of alkaline phosphatase, horseradish peroxidase, glucose oxidase, beta-galactosidase, urease, and combinations thereof.

24. A flow-through assay device as defined in claim 23, wherein said enzyme is horseradish peroxidase.

25. A flow-through assay device as defined in claim 22, wherein said redox label is used in conjunction with a microparticle modified with a specific binding member for the analyte.

26. A flow-through assay device as defined in claim 22, wherein said specific binding capture ligand has a specificity for the analyte at concentrations as low as about 10⁻⁹ moles of the analyte per liter of the test sample.

27. A flow-through assay device as defined in claim 22, wherein said calibration working electrode is formed from substantially the same material and has approximately the same shape and size as said detection working electrode.

28. A flow-through assay device as defined in claim 22, wherein a non-specific binding capture ligand is applied to said calibration working electrode.

29. A flow-through assay device as defined in claim 28, wherein said non-specific binding capture ligand has no specificity for the analyte at concentrations as high as about 10⁻² moles of the analyte per liter of the test sample.

30. A flow-through assay device as defined in claim 22, wherein a redox mediator is applied to said detection working electrode and said calibration working electrode.

31. A flow-through assay device as defined in claim 22, wherein the fluidic medium comprises a porous membrane or mesh.

32. A flow-through assay device as defined in claim 22, wherein the fluidic medium comprises a channel.

33. A method for detecting the presence or quantity of an analyte residing in a test sample, said method comprising:
i) providing a flow-through assay device comprising a fluidic medium in communication with an electrochemical affinity biosensor, said biosensor comprising a detection working electrode on which is immobilized a specific binding capture ligand for the analyte and a calibration working electrode;
ii) contacting a test sample containing the analyte with said fluidic medium;
iii) allowing the test sample to flow through said fluidic medium to contact said detection working electrode and said calibration working electrode;
iv) applying a potential difference between said detection working electrode and a counter electrode and between said calibration working electrode and a counter electrode;
v) measuring the current generated at the detection working electrode and the current generated at the calibration working electrode;
vi) determining a calibrated detection current by calibrating the current generated at the detection working electrode by the current generated at the calibration working electrode; and
vii) correlating the calibrated detection current to a concentration for the analyte.

34. A method as defined in claim 33, wherein a redox label is applied to said fluidic medium for directly or indirectly binding to the analyte.

35. A method as defined in claim 34, wherein said redox label is an enzyme selected from the group consisting of alkaline phosphatase, horseradish peroxidase, glucose oxidase, beta-galactosidase, urease, and combinations thereof.

36. A method as defined in claim 34, wherein said redox label is used in conjunction with a microparticle modified with a specific binding member for the analyte.

37. A method as defined in claim 33, wherein said specific binding capture ligand has a specificity for the analyte at concentrations as low as about 10⁻⁹ moles of the analyte per liter of the test sample.

38. A method as defined in claim 33, wherein said calibration working electrode is formed from substantially the same material and has approximately the same shape and size as said detection working electrode.

39. A method as defined in claim 33, wherein a non-specific binding capture ligand is applied to said calibration working electrode.

40. A method as defined in claim 39, wherein said non-specific binding capture ligand has no specificity for the analyte at concentrations as high as about 10⁻² moles of the analyte per liter of the test sample.

41. A method as defined in claim 33, wherein a redox mediator is applied to said detection working electrode and said calibration working electrode.

42. A method as defined in claim 33, wherein said potential difference is supplied by a multi-channel potentiostat.

43. A method as defined in claim 33, wherein the potential difference between said detection working electrode and said counter electrode is supplied simultaneously to the potential difference between said calibration working electrode and said counter electrode.

44. A method as defined in claim 33, wherein the current generated at the detection working electrode is measured simultaneously to the current generated at the calibration working electrode.

45. A method as defined in claim 33, wherein the fluidic medium comprises a porous membrane or mesh.

46. A method as defined in claim 33, wherein the fluidic medium comprises a channel.
